(19) United States
(12) Patent Application Publication
(10) Pub. No.: US 2017/0262940 A1
(43) Pub. Date: Sep. 14, 2017

(54) AUTOMATED MORTALITY CLASSIFICATION SYSTEM FOR REAL-TIME RISK-ASSESSMENT AND ADJUSTMENT, AND CORRESPONDING METHOD THEREOF

(71) Applicant: Swiss Reinsurance Company Ltd., Zurich (CH)

(72) Inventors: Thomas David MCCARTHY, Fort Wayne, IN (US); William Edward MOORE, Riverside, CT (US); Michael Bruce CLARK, Pound Ridge, NY (US); Jeffrey Stanton KATZ, Stamford, CT (US); Michael Wayne BERTSCHE, New Haven, IN (US); Edward Joseph WRIGHT, Fort Wayne, IN (US); Anand KANAKAGIRI, New Fairfield, CT (US); Pratik DAVE, Bethel, CT (US); Janis PENNER, Fort Wayne, IN (US)

(73) Assignee: Swiss Reinsurance Company Ltd., Zurich (CH)

(21) Appl. No.: 15/598,144

(22) Filed: May 17, 2017

Related U.S. Application Data
(63) Continuation of application No. PCT/EP2015/064904, filed on Jun. 30, 2015.

(51) Int. Cl. G06Q 40/08; G06Q 10/06

(52) U.S. Cl. CPC G06Q 40/08; G06Q 10/0635; G06Q 10/063; G06Q 40/02

(57) ABSTRACT

A system and method for real-time risk-assessment and adjustment, risks associated with a plurality of risk exposed individuals being at least partially transferable from a risk exposed individual to a first insurance system and/or from the first insurance system to an associated second insurance system. The system including a table with retrievable stored risk classes each comprising assigned risk class criteria, individual-specific parameters of the risk exposed individuals being captured relating to criteria of the stored risk classes by the system and stored in a memory, and a specific risk class associated with the risk of the exposed individual being identified out and selected of the stored risk classes by the system based on the captured parameters.

### CROSS-REFERENCE TO RELATED APPLICATIONS

This application is a continuation of International Application No. PCT/EP2015/064904 filed on Jun. 30, 2015; the entire contents of which are incorporated herein by reference.

### FIELD OF THE INVENTION

The present invention relates to automated mortality classification and signaling systems for real-time risk-assessment and adjustment. Based on the real-time risk-assessment and adjustment, specific risks associated with a risk-exposed individual are transferable from the risk-exposed individual to a first insurance system and/or from the first insurance system to an associated second insurance system, wherein the risk transfer is mutually synchronized between the first and second insurance system. The system comprises a table with retrievable stored risk classes each comprising assigned risk class criteria. Individual-specific parameters of the risk exposed individual are captured relating to criteria of the stored risk classes by means of the system and a specific risk class associated with the risk of the exposed individual is identified out and selected of said risk classes based on the captured parameters.

### BACKGROUND OF THE INVENTION

The problems associated with risk transfer and risk pooling are integral elements in the operation of life insurance systems, By grouping individuals' risk, the insurance systems are able to cover losses based on possibly future arising out of a common pool of resources captured by the insurance systems from associated individuals for the transfer of their risks. However, in order to maintain some degree of equity among individuals exhibiting different mortality risks, i.e. in order to derive a balance between a specific individual's risk transferred and the amount of its resources pooled in return, the insurance systems have to capture, assess and classified the individual's risk according to appropriately selected or filtered criteria and accepted characteristics. Historically, for a given risk transfer with a specific underwriting process. rates have been triggered by age and sex, and approximately nine-tenths of applicants have been accepted at the standard rote, and the rest has been individually rated or declined. However, from the late 1960s the insurance systems started to measure and consider more criteria, as e.g. non-smokers were charged lower rates. In the early 1990s, the process of adjusting the insurance systems' risk assessment further expanded to segregated sex and smoker-distinct lives employing other criteria. This continued segmentation of the standard class attempts to reduce mortality cross-subsidies in which better risks financially subsidize poorer risks, and thus refining and stabilizing the overall operation of the insurance systems. Each of a newer smaller class is expected to display a narrower distribution of mortality than the larger class from which it emerged. In this application, preferred lives or life risks are risks chosen according to measurable and triggerable criteria in addition to sex and tobacco use and which are expected to experience lower mortality as a group than the remaining non-rated lives of the same age, known as residual lives or residual lives risks or residual standard. Characteristic for so-called preferred life insurance systems is the generation of separate premium rates for preferred and residual live risks of the same age classified into two or more classes based on expected differing mortality.

A selected preferred group is expected to exhibit, on average, lower mortality than the residual group of individuals' risks. This does not imply that all preferred live risks have lower expected mortality than all residual lives, but that, when taken as a group, they can be expected to. At a most elementary level in the prior art, the preferred lives concept, implemented into insurance systems, divides the standard sex and smoker-distinct class into two classes by the use of certain admission criteria which are objectively defined and measured and which are known to be predictive of relative mortality. At the extreme end of the application of the preferred life insurance system, this method results in a unique rate charged a particular individual based on that individual's unique mortality risk profile. As long as death remains haphazard, the operational principle of the insurance systems is left intact. By removing any existent mortality cross-subsidy in the insured pool, i.e. the pooled individuals' risks by the insurance system, the most dissected, preferred life insurance systems represents the opposite of charging oil pooled, i.e. insured, individuals an identical rate, thereby balancing the risk over all associated individuals who's risk was transferred to the system. Therefore, such systems operate on complete risk transfer equity as opposed to complete risk transfer equality.

A critical point for the operational risk management for such insurance system typically involves consideration of one or more criteria, which are correlated to an event or events influencing the risk transferred. The ability to predict the frequency or eventual likelihood of occurrence of such critical events has value and utility in many settings. Often, different insurance systems use different sets of criteria to assess the expected occurrence of the same (or similar) events. In some cases, the same insurance systems may also use different criteria sets in differing situations or differing times. Methods and systems for comparing different criteria sets are useful tools in the selection of criteria and the design and development of related products. However, in the case of coupling the insurance system with a second insurance system, e.g. a mutually synchronized reinsurance system for seamless risk transfer within a negotiated parameter range, or in order to compare the products from competing insurance systems, or designing new preferred products to replace or augment existing products, the different operation of the insurance systems makes it difficult or even impossible to use methods which could take such differences into account. Clearly, such comparison may also be useful in the selection of criteria and pricing of related products, and in determining the impact of criteria changes or granting various exceptions to criteria on pricing and potential profitability of such products.

Another critical point for the operation of such insurance systems is complex process for assessing the appropriate risk of an individual on a mortality consistent basis. This is especially important for coupling a plurality of primary insurance systems to a second insurance system, i.e. reinsurance system, for hedging the operational risk and improve stability of the first insurance system by transferring the pooled individuals' risks at least partly to the second insurance system. Furthermore, incorporating the thinking process of underwriters during risk assessment and risk categorization is technically complex. In the prior art, there are different systems, disclosing an approach to the discussed problems. E.g. the patent U.S. Pat. No. 4,975,840 of A. DeTore et al. discloses a system for assessing the insurability of a potentially transferrable, i.e. insurable, risk, wherein the system comprises the ability to correlate selected elements of information in respective databases. Certain elements are assigned weights, as e.g. relative risk ratios, on the basis of predetermined relationships existing between elements of information in one database and corresponding elements of information in another. A risk classification is determined for the potentially insurable risk from the weights assigned. However, the weight must necessarily be assigned to a selected element based on the information in the databases manually, e.g. by an underwriter. For example, the underwriter typically must assign a risk classification based on their manual review of data and comparison with existing criteria. Further, the system is not able to provide an easy-to-use, real-time risk assessment by assessing the risks and classifying and/or categorizing the risks as technically required by preferred life insurance systems, though the systems comprises the ability to assign a different weight to an element of information, to use statistical profiles to adjust assigned weights, and the ability to determine expected profitably resulting from decisions concerning a particular risk in a manual manner. Thus this system is not able to manage and reduce the workload and customizing operation of the insurance system, as required. It is also not able to create an easy-to-use risk-assessment and risk-categorization instrument by automating and/or incorporating up-to-now necessarily, manually conducted processes. Further U.S. Pat. No. 6,456,979 of B. Flagg shows another system of the prior art for assessing the individuals' risks by establishing a benchmark cost of insurance value, obtaining a policy illustration for the transferred risk, resolving an cost of insurance value, and comparing the benchmark cost of insurance value with the illustrated cost of insurance value. Yet, another prior art system is disclosed by US 2003/0236685 of E. Buckner et al. In this system, for assessing the individual's risk, mortality data are electronically synthesized from a plurality of different insurance systems. A data engine processes the mortality data and synthesizes benchmark data to present the analyses. User inputs at remote computers are requested by the system, wherein these inputs are needed to process the risk assessment relative to one or more preferred life risk scenarios, such as age, height, weight, gender, blood pressure, cholesterol, familial cancer history, family history of heart attack, family history of stroke, smoker or non-smoker status, and smoking history.

However, all of the prior art systems are not able to completely solve the most important technical difficulties arise from capturing and assessing the risk that is associated with preferred incidents, i.e. the fully-automated and easy-to-operate risk assessment system. The prior art systems are not able to perform real-time risk assessment for preferred life insurance system. Moreover, they are not able to perform the risk-assessment on a mortality consistent basis. The capability of arriving of a precise measurement of an individual's preferred risk exposure is fundamental, inter alia, for the technical operation of risk-transfer systems or damage prevention/recovery systems, such as associated automated resource and risk pooling systems or automated insurance systems. The associated problem extends to the fact that the overall risk is typically spread over various single risks. Correspondingly, the different criteria and classes should be appropriately triggered. The overall associated or pooled risk cannot be captured or weighed on a automated bases by preferred life insurance systems as envisioned by the prior art providing an appropriate risk transfer.

### SUMMARY OF THE INVENTION

It is an object of the present invention to provide an automated real-time risk-assessment and adjustment system and method for measuring, accumulating and monitoring preferred life risks thereby providing a signaling system for transfer of specific risks associated with a risk-exposed individual from a first insurance system to an associated second insurance system based on the real-time risk-assessment and adjustment, which risk transfer is mutually synchronized between the first and second insurance system. Further, it is also an object of the present invention to provide a system and method for risk assessment and sharing of preferred life risks on a mortality consistent basis. It is a further object of the present invention to provide a system and method for real-time risk assessment and sharing of preferred life risks. It is another object of the present invention to provide a system and method allowing incorporate the thinking process of underwriters, refining the process and allowing for the best possible risk categorization on a completely automated basis.

According to the present invention, these objects are achieved, particularly, by the features of the independent claims. In addition, further advantageous embodiments con be derived from the dependent claims and related descriptions.

According to the present invention, the above-mentioned objects related to the measurement, accumulation and monitoring of preferred life risks are achieved, particularly, in that by means of a distinct-channel-based automated mortality classification system for real-time risk-assessment and adjustment, risks, which are associated with a plurality of risk exposed individuals, are at least partially transferred from a risk exposed individual to a first insurance system and/or from the first insurance system to an associated second insurance system, wherein the system comprises a table with retrievable stored risk classes each comprising assigned risk class criteria, wherein individual-specific parameters of the risk exposed individual are captured relating to criteria of the stored risk classes by means of the system and stored to a storage unit and wherein a specific risk class associated with the risk of the exposed individual is identified out and selected of said stored risk classes by means of the system based on the captured parameters, in that in a first channel, selectable by means of an user interface, to each of the risk classes of the table with retrievable stored risk classes, a first tolerance factor is determined and assigned to the corresponding risk class or user statements are posed for review, wherein in case that the captured parameter of a risk exposed individual fail to be matched to the criteria for one of the retrievable stored risk classes by means of the system, a relative mortality factor of the individual of the captured parameter is generated and compared to an average mortality of the closest matched class, and wherein based on the assigned first tolerance factor of the closest matched class, the system indicates whether to accept or reject a possible risk transfer for the individual for the closest matched class, in that in a second channel, selectable by means of the user interface, class category parameters with assigned criteria are defined comprising at least three class categories “standard”, “preferred” and “better preferred”, wherein a relative mortality factor is measured based on the captured individual's specific parameters and in relation to the average of expected mortality for the specific risk class associated with the individual, wherein a second tolerance factor for an excess mortality is determined and assigned to the corresponding risk class, and wherein the system indicates whether to assign the individual to the class category parameter “standard” or to a better class category “preferred” or “better preferred” based on the second tolerance factor, thereby providing the movement of the individual from class category “standard” to better class category, factors “preferred” and/or “better preferred”, and in that in a third channel, selectable by means of the user interface, individual-specific data are captured by the system, wherein the system triggers for predefined decline parameters in the captured individual-specific parameters, and upon detecting one of the predefined decline parameters the system declines a possible risk transfer for the individual for any class by transmitting appropriate decline data. It is important to note that the above given 3 class categories are just exemplary in number as well as in name definition. In the absolute minimum, the second channel must comprise at least two distinctive class categories otherwise there will be no reclassification mean. However, the second channel may also include more that three class categories, as for example 6 class categories (“standard”, “standard plus”, “preferred”, “preferred plus”, “better preferred”, “better preferred plus”), which allows for a more sophisticated and differentiated selection by means of the second channel. Such a second channel extended by more class categories, allow for a more distinctive movement between the class categories, and, thus, a more distinctive categorization, classification and recognition of the cases by means of the system. A more distinctive class categorization also enhances the power of the automated mortality classification system, technically providing the user or underwriter with an automated unique way to a more distinctive reclassification on a mortality consistent basis. Thus, improvement factors' that simulate the understanding process of underwriters, can be automatically captured by means of the system and refine the technical process allowing for the best possible risk categorization. Further, it is also important to note that better class category factors typically mean improved and thus betterclass category factors can, for example, correspond or be assigned by the system to lower premium or cost for the risk transfer, which is based on the lower morality associated with betterclass category factors. The risks associated with a plurality of risk exposed individuals can e.g. at least partially be transferable on a facultative basis by means of the automated mortality classification system from a risk exposed individual to a first insurance system and/or from the first insurance system to an associated second insurance system. Each of the risk classes of the table with retrievable stored risk classes can e.g. be associated to at least one financial product accessible in a dedicated data store, wherein the system determines, for each of the risk classes, an expected occurrence rate, wherein the system divides the expected occurrence rates by an average rate and determining a relative risk ratio as relative mortality factor for each of the risk classes based on the data relating to the criteria associated with said risk classes, wherein the system calculates correlated risk ratios between at least two of the risk classes that are identified in said step of identifying and determining a dependence between the at least two different risk classes, wherein the system compares the relative risk ratios and the correlated risk ratios with empirical data and generating comparative risk data to characterize the relative risks associated with the plurality of products, wherein the system corrects the relative risk ratios in a case the comparative risk data is out of a defined range comparing with the empirical data; and wherein an output device for outputting the corrected risk ratios. Further, the first channel can e.g. comprise a table with retrievable stored impairment criteria, wherein the first channel is only activatable in case of triggering at least one of the stored impairment criteria within the captured parameters of the risk exposed individual resulting in a failure to be matched to one of the risk classes, and wherein the table with retrievable stored impairment criteria comprises as trigger criteria for medical impairment criteria anemia, anxiety, asthma, atrial fibrillation and flutter, atrial septal defect, barrett's esophagus, bicuspid aortic valve, blood pressure, build, combination of blood pressure and lipids, combination of build and blood pressure, combination of build and lipids, crohn's disease, depression, diabetes mellitus type 2, epilepsy, mitral insufficiency, obstructive sleep apnea, rheumatoid arthritis, skin tumors other than melanoma, surgical treatment of obesity, thyroid, ulcerative colitis; comprises as trigger criteria for medical test criteria cholesterol/HDL ratio, EBCT, glomerular filtration rate (isolated elevation), EKG-T wave changes, impaired glucose tolerance, liver enzymes (isolated elevation), microalbuminuria (isolated elevation), proteinuria (isolated elevation), triglycerides; and comprises as trigger criteria for non-medical impairments aviation (private), driving, foreign travel, occupation, scuba diving. The second channel can e.g. comprise a table with retrievable stored improvement criteria, wherein the captured individual's specific parameters additionally comprise at least on of the retrievable stored improvement criteria, and wherein the improvement criteria comprise key preferred criteria associated with the core criteria, as build, blood pressure and treatment, cholesterol and HDL (High Density Lipoprotein) ratio and treatment, family history, as well as Improvement factors, as glycohemoglobin, statins treatment, prevention, wellness, NT-proBNP, ECG, stress test, and/or EBCT of the risk exposed individual. In the second channel, the at least three categories “standard”, “preferred” and “better preferred” refer to life-risks, which are either “standard”, i.e.

residual life-risks, i.e. not classified and therefore not captured by preferred life-risk insurance systems, or satisfying the condition of one preferred criterion, i.e. a “preferred” life-risk captured by preferred life-risk insurance systems, or even satisfying two or more criteria of preferred life-risk insurance, i.e. getting an even better rating as only “preferred” life-risks. Again, the three class categories just serve as common example.

The technical design of the second channel is neither limited to three criteria nor to the above chosen naming as “standard”, “preferred” and “better preferred”. The invention has, inter alia the advantage that it allows for the implementation of an automated system, for a scenario-based life-risk determination of risk exposure of a risk-exposed component, i.e. individual, and/or the risk-assessment of the overall risk transferred and pooled by the insurance system by means of the segmented and weighted accumulation of the various exposures in a preferred risk-pooling mode. The invention allows measuring, accumulating and monitoring preferred life risks in a distinct and controllable way. Further, the present invention has the advantage to be capable of providing the technical requirements for risk assessment and sharing of preferred life risks on a mortality consistent basis and in a fully automated way. Further invention has the advantage to provide a real-time system and method for real-time risk assessment and sharing of preferred life risks. Finally, present invention allows incorporating and simulating the thinking process of underwriters, thereby refining the process and allowing for the best possible risk categorization on a completely automated basis.

In one embodied variant for measuring, accumulating and monitoring preferred life risks, by triggering predefined decline parameter in the captured individual-specific parameters, additional individual-specific data are requested by the system and transmitted to an independent review unit, wherein only upon capturing the transmission of a check back confirmation of the review unit, the system declines a possible risk transfer for the individual for any class by transmitting appropriate decline data. This embodied variant has, inter alia, the advantage that the third channel has additional flexibility built in to allow the first insurance system and/or a risk exposed individual to either add freeform comments related to an impairment and submit to the second insurance system for review or select freeform without an impairment, add comments and submit to an associated second insurance system's underwriting system for review and control.

In an other embodied variant, the system comprises an otional extension of the third channel, which is realized as a freeform aspect of the third channel extending the case capturing capability of the third channel accordingly, and which is selectable by means of the user interface in case that the captured parameters of a risk exposed individual are not yet classified, the risk exposed individual is classified based upon matching to the criteria for one of the retrievable stored risk classes by means of the system, wherein the first and/or second and/or third channel are only selectable by means of an user interface, if the captured parameters of a risk exposed individual fail to be matched to the criteria for one of the retrievable stored risk classes by means of the system. This embodied variant has, inter alia, the advantage that the system allows a complete real-time risk assessment measurement, and monitoring of preferred life risks without any human interaction.

As a further embodied variant, the system comprises as fourth channel, a further channel selectable by means of the user interface in case that the captured parameters of a risk exposed individual are not yet classified, the system provides, in response on selection, input means for a facultative case summary submission for the new individual, the input means prompting for a new individual of the first insurance system, e.g. associated with a ceding company, to enter facultative case summary information including risk factor information for evaluating a risk in providing risk cover by means of the second insurance system for the new individual. As response to the received input, that system renders by means of the fourth channel a facultative decision based on the received facultative case summary submission. As a variant to this fourth channel, the input means further prompt for a new individual of the first insurance system to identify at least one second insurance system to receive the facultative case summary information. The system receives for the new individual of the first insurance system, over the network, the facultative case summary information and a plurality of second insurance systems, i.e. reinsurers, identified by the first insurance system to receive the facultative case summary information. The system stores the received facultative case summary information and the plurality of identified second insurance systems in a memory module. In the following, the system provides the stored facultative case summary information over the network of the plurality of identified second insurance systems, the facultative case summary information being used by the plurality of identified second insurance systems to render facultative decisions on whether to provide second risk cover, i.e. reinsurance, for the newly submitted facultative case summary information. Also in the latter variant of the fourth channel, the risk exposed individual is classified based upon matching to the criteria for one of the retrievable stored risk classes by means of the system, wherein the first and/or second and/or third channel are only selectable by means of an user interface, if the captured parameters of a risk exposed individual fail to be matched to the criteria for one of the retrievable stored risk classes by means of the system. As a variant, the input step can also comprises receiving from the first insurance system a selection of a type of case to be submitted. This embodiment variant has inter alia the advantage that this inventive case summary facultative underwriting channel, i.e. the fourth channel, overcomes the prior art problem that every document in the case history must be reviewed before rendering a facultative decision. This embodiment variant has further the advantage that the invention provides a system for first insurance systems to submit case summary information over a network for review by one or more second insurance systems thereby increasing consistency, providing faster review, and greater convenience for underwriters of the ceding company. Thus, the fourth channel allows for processing a life insurance facultative case summary submission over a network between a first insurance system and a second insurance system, wherein initially by means of the fourth channel, a facultative case summary submission is received by the second insurance system from the first insurance system, via the network, and thereafter, a facultative decision is rendered by the inventive system itself and/or the second insurance system based on the received facultative case summary submission.

Because the information is summarized and sent electronically, less information is processed in a faster period of time thereby rendering quicker decisions than when the complete case history is submitted to the second insurance system for review.

In a further embodied variant, risks associated with a plurality of risk exposed individuals are at least partially transferable from a risk exposed individual to a first insurance system and/or from the first insurance system to an associated second insurance system by means of the automated mortality classification system or a Life Mortality System (LMS), wherein an appropriate activation signaling is generated by the automated mortality classification system or the Life Mortality System and transmitted to the first insurance system and/or to the associated second insurance system. This embodied variant has, inter alia, the advantage that the risk transfer can be fully automated, controlled and monitored by means of the system.

In another embodied variant, the number of pooled risk exposure components is dynamically adapted, by means of the resource-pooling system within the automated mortality classification system or the Life Mortality System, to a range where non-covariant, occurring risks covered by the resource-pooling system affect only a relatively small proportion of the total pooled risk exposure components at any given time. This variant has, inter alia, the advantage that it helps to improve the operational and financial stability of the system.

In a further embodied variant, the criteria and/or related measuring parameters are dynamically adapted by means of an operating module based on time-correlated incidence data for a preferred life risk condition indicating changes in the condition of the risk component, i.e. the corresponding individual. This variant has, inter alia, the advantage that changings or new occurrence in the criteria or in measurements of the criteria, condition and/or boundary parameters can be dynamically captured by the system and dynamically affect the overall operation of the system based on the risk of the pooled risk exposure of the risk exposed individual.

In an embodied variant, the system comprises means to automatically negotiate the risk class criteria between the first insurance system and second insurance system. This variant has, inter alia, the advantage that the system, and especially the coupling of the first and the second insurance system can be more flexible, and moreover dynamically adapted by the first and second insurance system.

In an even further embodied variant, said one or more risk classes can be associated with one or more criteria, and the system further modifies one or more of said criteria and re-determining the relative risk ratio and for determining on impact of said modification on the relative risks associated with the products. One or more of said risk classes can e.g. be associated with different criteria, and the system further compares the risk classes based on said relative risk ratios. Further, the system can e.g. redefine one or more of said risk classes based on the relative risk ratio. As a variant, the system also can determine a separate relative risk ratio for sub-groups of risks. The system also can e.g. compare the prevalence data to industry empirical data for particular combinations of criteria; and adjusts the stored data to agree with the empirical data. All these variants have, inter alia, the advantage that they allow to further improve the operation and the operational stability of the system during operation. Further they allow a more precise risk assessment for the pooling of preferred life risks.

In yet another embodied variant, upon each triggering of an occurrence of measuring parameters indicating one of the predefined life risks, a non-parametric payment or a total parametric payment is allocated with this triggering, and wherein the total allocated payment is transferrable upon the triggering of the occurrence of the life risk. This has inter alia the advantage that the inventive system is able to set amount limits, e.g. minima and maxima, for various first insurance systems and/or risk exposed individuals. In the embodied variant of the parametric payment, the payment can be leveled with regard to a predefined total payment sum that is determined at least based on the risk-related individual's data, and/or on the likelihood of the risk exposure for one or a plurality of the pooled risk exposed individuals based on the risk-related data. The predefined total payments can e.g. be leveled to any appropriate lump sum or any other sum related to the total transferred risk and the amount of the periodic payments of the risk exposure component. This variant has, inter alia, the advantage, the overall operation of the real-time automated mortality classification system together with the first and/or second insurance system can be fully automated. Further, the parametric variant has the advantage, inter alia, that the transfer of the payment by the automated insurance system, which depends on the measuring of an occurrence of a life risk event, allows for an adapted payment of the total sum that is dependent on the determined impact of life risk event on the risk exposed individual. In one embodied variant, a periodic payment transfer from the risk exposure individual to a resource pooling system of the first insurance system via a plurality of payment receiving modules is requested by means of a monitoring module of the resource-pooling system, and wherein the risk transfer or protection for the risk exposure individual is interrupted by the monitoring module, when the periodic transfer is no longer detectable by means of the monitoring module. As a variant, the request for periodic payment transfers can be interrupted automatically or waived by means of the monitoring module, when the occurrence of indicators for a life risk event is triggered in a data flow pathway associated with a risk exposed individual. These embodied variants have, inter alia, the advantage that the system allows for a further automation of the monitoring operation, especially of its operation with regard to the pooled resources. In addition, an independent verification trigger of the first or second insurance system can e.g. be activated in cases of a triggering of the occurrence of indicators for a life risk event in the data flow pathway of a risk exposed individual by means of the trigger module, and wherein the independent verification trigger, additionally, is triggering for the occurrence of indicators regarding the concerned life risk event in an alternative data flow pathway with independent measuring parameters from the primary data flow pathway of the individual in order to verify the occurrence of the life risk event at the risk exposed individual. As a variant, the transfer of payments is only assigned to the corresponding risk exposed individual if the occurrence of the life risk event at the risk-exposed individual is verified by the independent verification trigger. These embodied variants have, inter alia, the advantage that they help improve the operational and financial stability of the first and second insurance system. in addition, the system is rendered less vulnerable relative to fraud and counterfeit.

Finally, in addition to the system, as described above, and the corresponding method, the present invention also relates to a computer program product that includes computer program code means for controlling one or more processors of the control system in such a manner that the control system performs the proposed method; and it relates, in particular, to a computer program product that includes a computer-readable medium containing therein the computer program code means for the processors.

### BRIEF DESCRIPTION OF THE DRAWINGS

The present invention will be explained in more detail by way of example in reference to the drawings in which:

FIG. 1 shows a block diagram illustrating schematically an exemplary automated mortality classification and facultative system 1 for automated assessment and measurement of preferred life risks with a distinct channel-based scheme 21,22,23 with the optional extension 231 of the third channel 23 realized as a freeform aspect of the third channel, and the optional fourth channel 24 extending the caturing and processing capability of the system 1 to capturing and processing facultative case summary submission.

FIG. 2/3/4 show further block diagrams illustrating schematically an exemplary automated mortality classification and facultative system 1 for automated assessment and measurement of preferred life risks.

FIG. 5 shows a block diagram illustrating schematically the generation of classified life-risk out of a cohort of unclassified (standard) life-risk. Members of the preferred part show a measurable lower relative mortality that the members of the standard part, Unclassified life-risks can be by default categorized as standard life-risks.

FIG. 6 shows a block diagram illustrating schematically the generation of classified life-risk out of a cohort of unclassified (standard) life-risk. The life-risks matching one or more of the preferred criteria are classified or reclassified from standard category to preferred or better-preferred category. In FIG. 6, during classification, the overall standard cohort (dotted line) is divided into two classes, wherein each class comprises sufficient live-risks that their cohort follows a normal distribution, with proper means and (smaller, or less volatile) standard deviations. Based on the characteristics of the normal distribution, the overlap between the two classes does not imply a misclassification of individual life-risks by the system but naturally and necessarily occurs indicating the proper operation of the classification procedure.

FIG. 7 shows a block diagram illustrating schematically a part of the operational workflow in the exemplary automated mortality classification and facultative system 1 for automated assessment and measurement of preferred life risks with a distinct channel-based scheme 21,22,23. The optional freeform third channel extension 231 is dependent of the realization redundant due to the operation of the third decline channel 23. Further, the optional fourth channel 24 extends the catering and processing capability of the system 1 to capturing and processing facultative case summary submission.

### DETAILED DESCRIPTION OF THE PREFERRED EMBODIMENTS

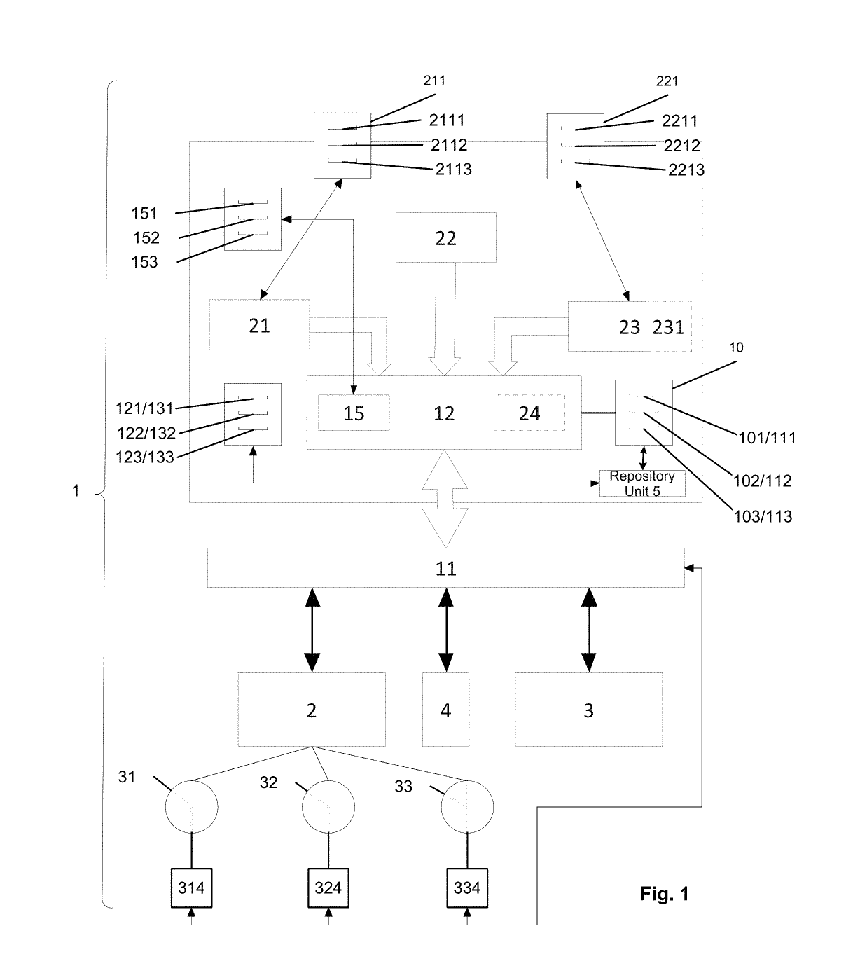

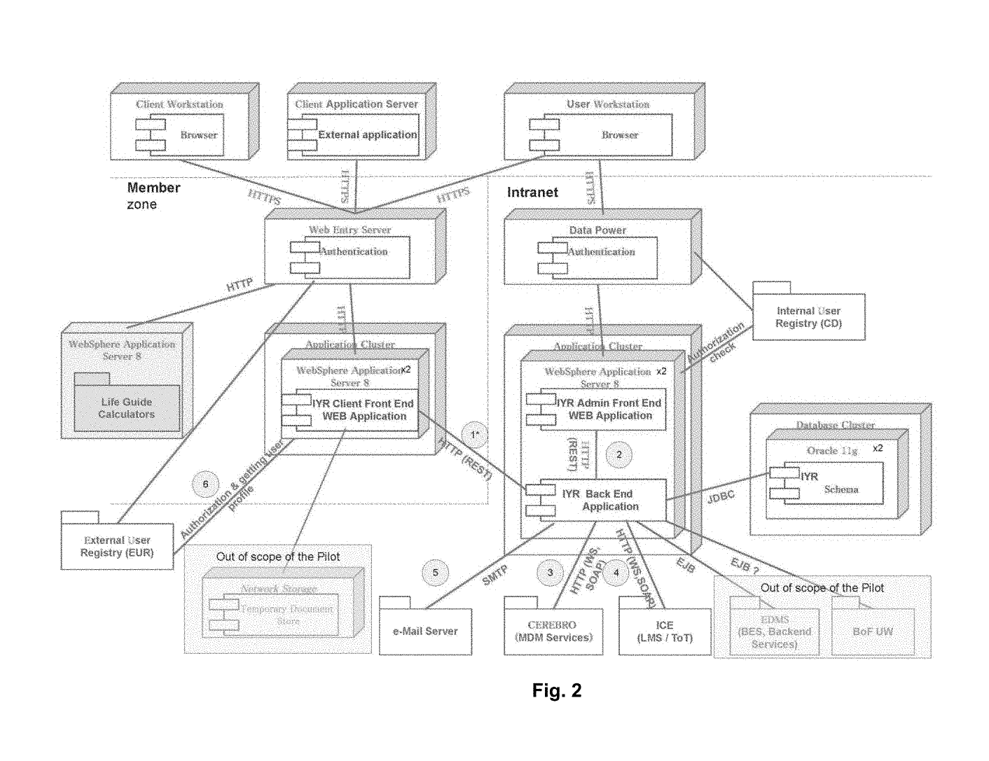

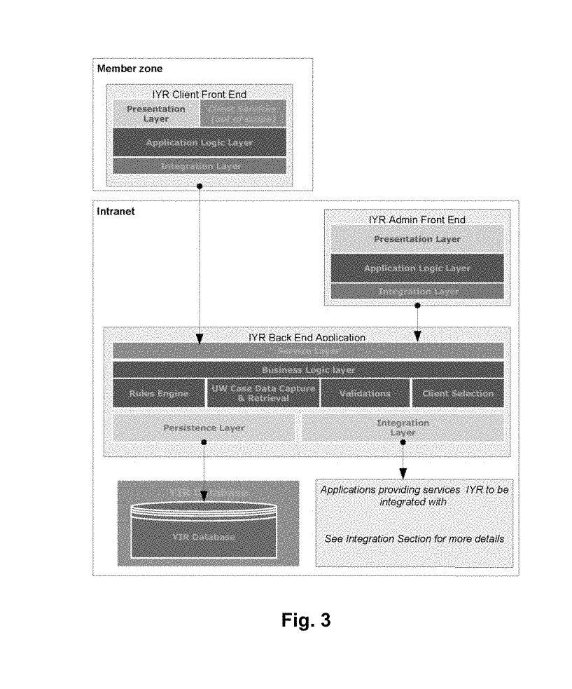

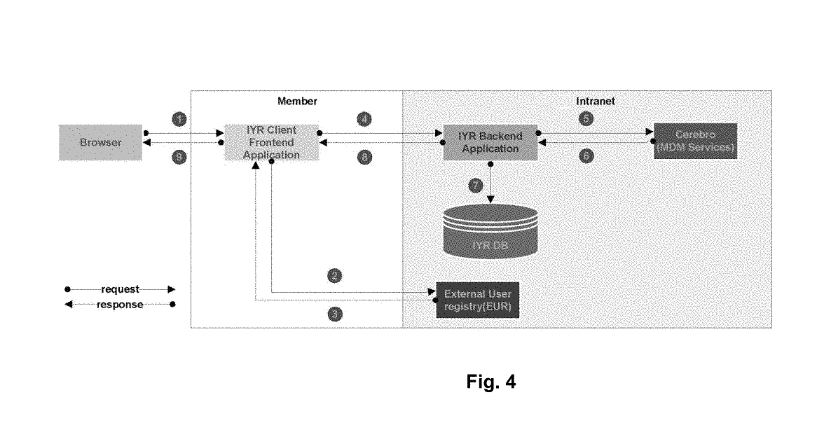

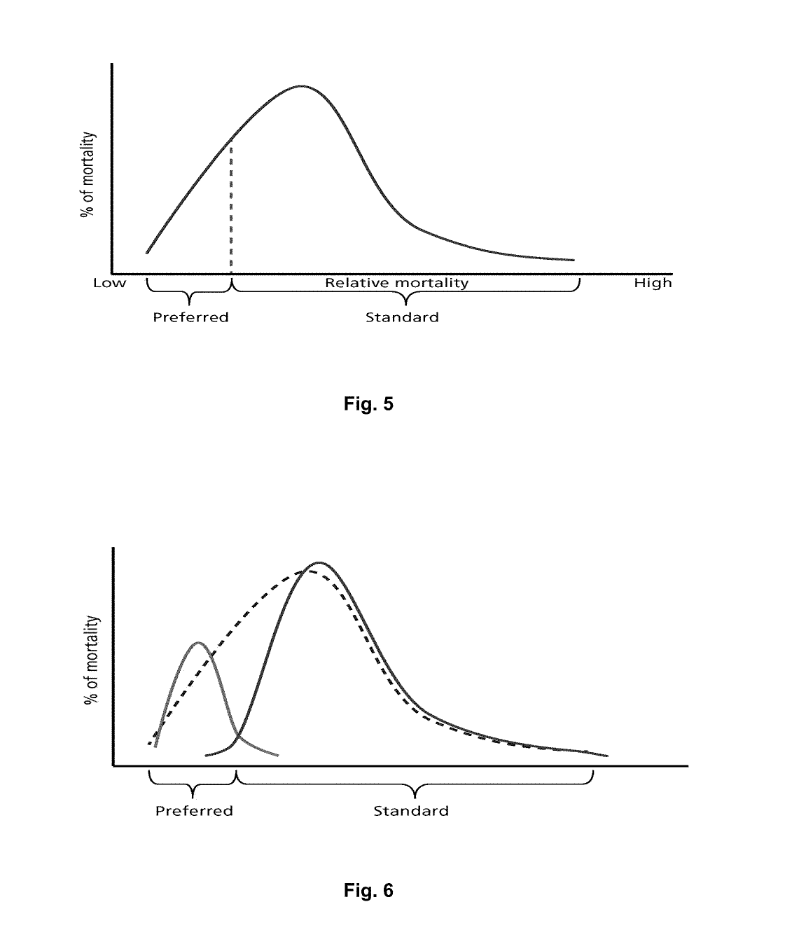

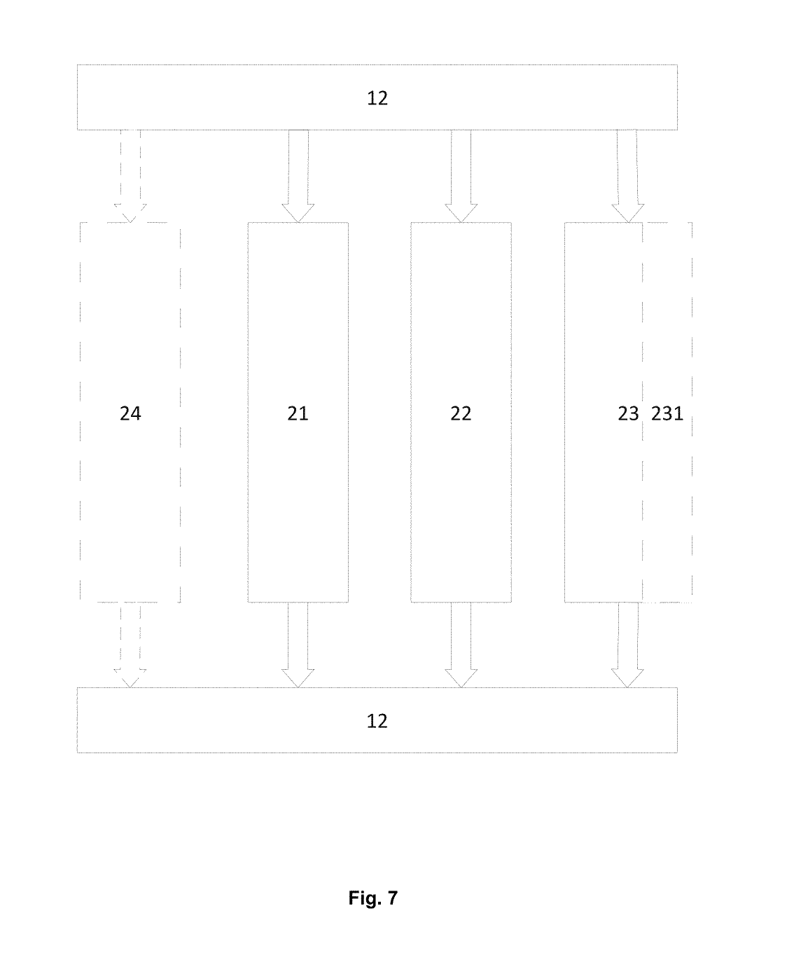

FIG. 1 illustrates, schematically, an architecture for a possible implementation of an embodiment of the automated mortality classification and facultative system 1 for measurement and accumulation of life risks, as well as an architecture for a possible implementation of on embodiment of an automated life-risk insurance system based on a resource-pooling system for risk sharing of life risks of a variable number of risk exposed individuals 31, 32, 33. Resource-pooling systems 2/3 are systems for automated pooling of resources from assigned risk exposed individuals 31, 32, 33, thereby transferring a defined risk associated with the risk exposed individuals 31, 32, 33, to the resource-pooling systems 2/3, wherein the operation of the transferred risk is defined by risk-transfer parameters, cis e.g. fixed by means of predefined risk-transfer policies, and wherein in case of triggering the occurrence of the defined life risk at a risk exposed individual 31, 32, 33, . . . , a loss of the concerned risk exposed individual 31, 32, 33, . . . is distinctively covered by the resource-pooling systems 2/3 by triggering the specific transfer of resources from the resource-pooling system 2/3 to the concerned risk exposed individual 31, 32, 33, . . . The operation of the system 1 will be described in detail below. The risk-transfer parameters can e.g. comprise parameters defining physical measuring parameters to detect the occurrence of a risk event at the risk exposed individual 31, 32, 33, by means of the system 1 and/or time- or amount related threshold values. The risk exposed individuals 31, 32, 33, . . . can be any type of person associated with a life risk. A life risk is related to the probability for the occurrence of a life risk event in relation to risk-exposed individuals 31, 32, 33. The automated mortality classification system 1 includes at least a processor and associated memory modules. The operation of the system 1 is controlled, monitored and steered by the control module 12, in particular generating appropriate signaling and steering the activation and interworking of the various components of the automated mortality classification system 1. The automated mortality classification system 1 can also include one or more display units and operating elements, such as a keyboard, and/or graphic pointing devices, such as a computer mouse. The system 1 is a technical device comprising electronic means that can be used in the field of automated risk transfer or insurance technology with regard to risk transfers that are related to life risks. The invention seeks to technically capture, manage and automate complex related operations of monitoring devices and the insurance industry. Another aspect involves synchronizing and adjusting such operations based on the proposed technical means. In contrast to standard approaches, the resource-pooling system also achieves reproducible operations and reproducible risk assessment with the desired technical, repetitious accuracy, because it relies completely on technical means, process flow and process control/operation. The automated mortality classification system 1 provides a scenario-based, life-risk measurement and determination of the risk exposure of risk-exposed individuals 31, 32, 33, . . . or of an insurance portfolio containing a plurality of risk-exposed individuals 31, 32, 33, . . . by means of the weighted accumulation of the various exposures associated with the corresponding risk classes 101, 102, 103 and risk class criteria 110, 111, 112. For the risk assessment, a distinct channel-based operation scheme is applied using appropriate life risk segmentation. The total or maximum exposure is derived by summing up the different exposures per risk class over all accumulated classes, or contracts/treaty contracts in a portfolio.

The distinct channel-based automated mortality classification system 1 for automated real-time risk-assessment and adjustment of preferred life-risks captures life-risks associated with a plurality of risk exposed individuals 31, 32, 33, which risks are at least partially transferable from a risk exposed individual 31, 32, 33 to a first insurance system 2 and/or from the first insurance system 2 to an associated second insurance system 3. The risks associated with a plurality of risk exposed individuals 31, 32, 33 can e.g. be at least partially transferable on a facultative basis by means of the automated mortality classification system 1 from a risk exposed individual 31, 32, 33 to a first insurance system 2 and/or from the first insurance system 2 to an associated second insurance system 3. The risks associated with the plurality of risk exposed individuals 31, 32, 33 can e.g. be at least partially transferable from a risk exposed individual 31, 32, 33 to a first insurance system 2 and/or from the first insurance system 2 to an associated second insurance system 3 by means of the automated mortality classification system 1, wherein an appropriate activation signaling is generated by the automated mortality classification system 1 and transmitted to the first insurance system 3 and/or to the associated second insurance system 3 in order to activate and/or execute the risk-transfer. The system 1 comprises a table 10 with retrievable stored risk classes 101, 102, 103 each comprising assigned risk class criteria 110, 111, 112. Each of the risk classes 101, 102, 103 of the table 10 with retrievable stored risk classes 101, 102, 103 can e.g. be associated to at least one financial product accessible in a dedicated data store. The system 1 determines, for each of the risk classes 101, 102, 103, an expected occurrence rate, wherein the system 1 divides the expected occurrence rates by an average rate and determining a relative risk ratio as relative mortality factor for each of the risk classes 101, 102, 103 based on the data relating to the criteria 110, 111, 112 associated with said risk classes 101, 102, 103. The system 1 calculates correlated risk ratios between at least two of the risk classes 101, 102, 103 that are identified in said step of identifying and determining a dependence between the of least two different risk classes 101, 102, 103. The system 1 compares the relative risk ratios and the correlated risk ratios with empirical data and generating comparative risk data to characterize the relative risks associated with the plurality of products, wherein the system 1 corrects the relative risk ratios in a case the comparative risk data is out of a defined range comparing with the empirical data. In this case, the system 1 can e.g. comprise an output interface 11 for outputting the corrected risk ratios. Thus, certain embodiment features of the present invention are directed to assessing relative risks, such as mortality risks, for a plurality of financial products, such as preferred insurance products. This can comprise the steps of (i) identifying one or more risk classes 101, 102, 103 associated with the plurality of products, (ii) determining for each of the risk classes 101, 102, 103 an expected occurrence rate, (iii) dividing the expected rates by an average rate for standard risks to determine a relative risk ratio for each of the risk classes 101, 102, 103, and (iv) comparing the relative risk ratios to characterize the relative risks associated with the plurality of products. Finally, concerning the criteria the automated mortality classification system 1 can e.g. comprise means to automatically negotiate the risk class criteria 110, 111, 112 between the first insurance system 2 and second insurance system 3. This allows c further level of automation of the overall operation of the system.

The individual-specific parameters of the risk exposed individuals 31, 32, 33 are captured relating to criteria 110, 111, 112 of the stored risk classes 101, 102, 103 by means of the system 1 and/or capturing or measuring devices 314, 324, 334, and stored to a storage or repository unit 5. A specific risk class 101, 102, 103 associated with the life-risks of the exposed individual 31, 32, 33 is identified out and selected of said stored risk classes 101, 102, 103 by means of the system 1 based on the captured parameters.

In a first channel 21, selectable by means of an user interface 11, to each of the risk classes 101, 102, 103 of the table 10 with retrievable stored risk classes 101, 102, 103, a first tolerance factor is determined and assigned to the corresponding risk class 101, 102, 103. In case that the captured parameters of a risk exposed individual 31, 32, 33 fail to be matched to the criteria 110, 111, 112 for one of the retrievable stored risk classes 101, 102, 103 by means of the system 1, a relative mortality factor of the individual 31, 32, 33 of the parameters is generated and compared to an average mortality of the closest matched class. Based on the assigned first tolerance factor of the closest matched class 101, 102, 103, the system 1 indicates whether to accept or reject a possible risk-transfer for the individual 31, 32, 33 for the closest matched class 101, 102, 103. The first channel 21 can e.g. comprise a table 211 with retrievable stored impairment criteria 2111, 2112, 2113, wherein the first channel 1 is only activatable in case of triggering at least one of the stored impairment criteria 2111, 2112, 2113 within the captured parameters of the risk exposed individuals 31, 32, 33 resulting in a failure to be matched to one of the risk classes 101, 102, 103. The table 211 with retrievable stored impairment criteria 2111, 2112, 2113 can e.g. comprise as trigger criteria for medical impairment measuring parameters anemia, anxiety, asthma, atrial fibrillation and flutter, atrial septal defect, barrett's esophagus, bicuspid aortic valve, blood pressure, build, combination of blood pressure and lipids, combination of build and blood pressure, combination of build and lipids, crohn's disease, depression, diabetes mellitus type 2, epilepsy, mitral insufficiency, obstructive sleep apnea, rheumatoid arthritis, skin tumors other than melanoma, surgical treatment of obesity, thyroid, ulcerative colitis; comprises as trigger criteria for medical test criteria cholesterol/HDL ratio, EBCT, glomerular filtration rate (isolated elevation), EKG-T wave changes, impaired glucose tolerance, liver enzymes (isolated elevation), microalbuminuria (isolated elevation), proteinuria (isolated elevation), triglycerides; and comprises as trigger criteria for non-medical impairments aviation (private), driving, foreign travel, occupation, scuba divine.

In a second channel 22, selectable by means of the user interface 11, class category parameters 121, 122, 123 with assigned class category criteria 131, 132, 133 are defined comprising at least three class categories “standard”, “preferred” and “better preferred”. A relative mortality factor is measured based on the captured individual's specific parameter and the class category criteria 131, 132, 133 and in relation to the average of expected mortality for the specific risk class 101, 102, 103 associated with the individual 31, 32, 33. A second tolerance factor for an excess mortality is determined and assigned to the corresponding risk class 101, 102, 103. The automated mortality classification system 1 indicates whether to assign the individual 31, 32, 33 to the class category parameter 121, 122, 123 “standard” or to a better class category, i.e. “preferred” or “better preferred”, based on the second tolerance factor, thereby providing the movement of the individual 31, 32, 33 from class category “standard” to better class category factors “preferred” and/or “better preferred”. The second channel 22 can e.g. comprise a table 221 with retrievable stored improvement criteria 2211, 2212, 2213, wherein the captured individual's specific parameters additionally comprise at least on of the retrievable stored improvement criteria 2211, 2212, 2213. The improvement criteria 2211, 2212, 2213 can e.g. comprise key preferred measuring parameters associated with the build, blood pressure and treatment, cholesterol and ratio and treatment, family history, glycohemoglobin, statins treatment, prevention, wellness, NT-proBNP, ECG, stress test, and/or EBCT of the risk exposed individual 31, 32, 33. In the second channel, the categories “standard”, “preferred” and “'better preferred” refer to life-risks, which are either “standard”, i.e. residual life-risks, i.e. not classified and therefore not captured by preferred life-risk insurance systems, or satisfying the condition of one preferred criterion, i.e. a “preferred” life-risk captured by preferred life-risk insurance systems, or even satisfying two or more criteria of preferred life-risk insurance, i.e. getting an even better rating as only “preferred” life-risks.

For both, first and second channel 21/22, said one or mare risk classes 101, 102, 103 can e.g. be associated with one or more risk class criteria 110, 111, 112, and the system 1 further modifies one or more of said criteria 110, 111, 112 and re-determining the relative risk ratio and for determining an impact of said modification on the relative risks associated with the products. The one or more risk classes 101, 102, 103 can e.g. be associated with different criteria 10, 111, 112, and the system further compares the risk classes 101, 102, 103 based on said relative risk ratios. For example, the system 1 can, as embodiment variant, also redefine one or more of said risk classes 101, 102, 103 based on the relative risk ratio. Further, system 1 can determine a separate relative risk ratio for sub-groups of risks. Finally, it is also possible that the system 1 further compares the prevalence data to industry empirical data for particular combinations of risk class criteria 110, 111, 112 and adjusts the stored data to agree with the empirical data.

In a third channel 23, selectable by means of the user interface 11, individual-specific parameter are captured by the system 1 by means of the interface 11 or capturing or measuring devices 314, 324, 334. The automated mortality classification system 1 triggers for predefined decline parameters 151, 152, 153 in the captured individual-specific parameter, and upon detecting one of the predefined decline parameters 151, 152, 153, the system 1 declines a possible risk-transfer for the individual 31, 32, 33 for any of the risk classes 101, 102, 103 by transmitting appropriate decline data. As a embodiment variant in case of triggering predefined decline parameter 151, 152, 153 in the captured individual-specific data, additional individual-specific parameters can be requested by the system 1 and transmitted to an independent review unit 4, wherein only upon capturing the transmission of a check back confirmation of the review unit 4, the automated mortality classification system 1 declines a possible risk transfer for the individual 31, 32, 33 for the classes 31, 32, 33) by transmitting appropriate decline data.

Finally, as a further embodiment variant, the automated mortality classification system 1 comprises optional extension channel 231 of the third channel 23, which is realized as a freeform aspect of the third channel 23 extending the case capturing capability of the third channel 23 accordingly, and which is selectable by means of an user interface 11 in case that the captured parameters of a risk exposed individual 31, 32, 33 are not yet classified, the risk exposed individual 31, 32, 33 is classified based upon matching to the parameters for one of the retrievable stored risk classes by means of the system 1. In this case, the first 21 and/or second 22 and/or third 23 channel can for example only be activated by the automated mortality classification system 1, i.e. selectable by means of an user interface 11, if the captured parameters of a risk exposed individual 31, 32, 33 fail to be matched to the criteria 110, 111, 112 for one of the retrievable stored risk classes 101, 102, 103 by means of the automated mortality classification system 1. As an embodiment variant, the system 1 can further comprise a trigger module 15. The trigger module 15 can be connected to the risk components 31, 32, 33, . . . by means of capturing devices 314, 324, 334 in order to detect and capture measuring values for the captured parameters related to the occurrence of life risk events within the data pathway associated with a risk exposed individual 31, 32, 33, . . . . The data flow pathway can e.g. be monitored by the system 1, capturing individual-related measuring parameters at least periodically and/or within predefined time periods. The data flow pathway can, for example, also be dynamically monitored by the automated mortality classification system 1 and/or one of the insurance systems 2/3, by triggering individual-measuring parameters of the data flow pathway transmitted from associated measuring systems. Triggering the data flow pathway, which comprises dynamically recorded measuring parameters of the concerned risk exposed individuals 31, 32, 33, the system 1 is also able to detect the occurrence of a life risk event and dynamically monitor the different stages throughout the occurrence of the life risk event in order to provide appropriately adapted and gradated risk protection for a concerned risk exposed individual 31, 32, 33, Such a risk protection structure can be based on received and stored payments from the related risk exposed individual 31, 32, 33, . . . , and/or related to the total risk of the insurance system 2 or 3 based on the overall transferred life risks of all pooled risk exposed individuals 31, 32, 33.

As an other optional variant, the system 1 may comprise as fourth channel 24, a further channel 24 selectable by means of the user interface 11 in case that the captured parameters of a risk exposed individual 31, 32. 33 are not yet classified, the system 1 provides, in response on selection, input means for a facultative case summary submission for the new individual 31, 32, 33, the input means prompting for a new individual of the first insurance system 2, e.g. associated with a ceding company, to enter facultative case summary information including risk factor information for evaluating a risk in providing risk cover by means of the second insurance system 3 for the new individual 31, 32, 33, The prompted input means, e.g. realized, as part of the data interface 11, can for example comprise a webpage-based input options or other appropriate data entry means. As response to the received input, that system 1 renders by means of the fourth channel 24 a facultative decision based on the received facultative case summary submission. As a variant to this fourth channel 24, the input means further prompt for a new individual 31, 32, 33 of the first insurance system 2 to identify at least one second insurance system 2 to receive the facultative case summary information. The system 1 receives for the new individual 31, 32, 33 of the first insurance system 2, over the network, the facultative case summary information and a plurality of second insurance systems 3, e.g. associated with reinsurers, identified by the first insurance system 2 to receive the facultative case summary information. The system 1 stores the received facultative case summary information and the plurality of identified second insurance systems 3 in a memory module. In the following, the system provides the stored facultative case summary information over the network of the plurality of identified second insurance systems 3, the facultative case summary information being used by the plurality of identified second insurance systems 3 to render facultative decisions on whether to provide second risk cover, i.e. risk transfer for reinsurance, for the newly submitted facultative case summary information. Also in the latter variant of the fourth channel 24, the risk exposed individual 31, 32, 33 is classified based upon matching to the criteria 110, 111, 112 for one of the retrievable stored risk classes 101, 102, 103 by means of the system, wherein the first and/or second and/or third channel are only selectable by means of an user interface 11, if the captured parameters of a risk exposed individual 31, 32, 33 fail to be matched to the criteria for one of the retrievable stored risk classes 101, 102, 103 by means of the system 1. As a variant, the input step can also comprises receiving from the first insurance system 2 a selection of a type of case to be submitted. This embodiment variant has inter alia the advantage that this inventive case summary facultative underwriting channel 24, i.e. the fourth channel 24, overcomes the prior art problem that every document in the case history must be reviewed before rendering a facultative decision. This embodiment variant has further the advantage that the invention provides an automated system for first insurance systems 1 to submit case summary information over a network for review by one or more second insurance systems 3 thereby increasing consistency, providing faster review, and greater convenience for underwriters of the ceding company. Thus, the fourth channel 24 allows for processing a life insurance facultative case summary submission over a network between a first insurance system 2 and a second insurance system 3, wherein initially by means of the fourth channel 24, a facultative case summary submission is received by the second insurance system 3 from the first insurance system 1, via the network, and thereafter, a facultative decision is rendered by the inventive system itself and/or the second insurance system 3 based on the received facultative case summary submission. Because the information is summarized and sent electronically, less information is processed in a faster period of time thereby rendering quicker decisions than when the complete case history is submitted to the automated second insurance system 2 for review.

In the first and second channel 21/22, for example, the individual risks can be mortality risks, and more specifically mortality risks, which are, based on a plurality of preferred risk criteria 110, 111, 112. Thus, these channels 21/22 can be used to compare and evaluate preferred risk classifications used by different insurance systems in connection with their respective products. Different criteria 110, 111, 112 are often used as operational parameters of different, automated insurance systems 2/3 in determining which risks are selectable to be preferred. The embodiment illustrated in the FIG. 1 allows also for comparison of preferred insurance products, notwithstanding the differences in the preferred criteria used by different companies. However, most important, the risk assessment in the first and second channel 21/22 allows to put the different systems on a mortality consistent basis.

It is to be noted, that as a part of putting the automated mortality classification system 1 in operation, the system 1 comprises capturing or determining the rate of occurrence of a criterion (or criteria) 110, 111 112 among the risk exposed individuals 31, 32, 33 (or broader an insured population) to be captured by the system 1. This rate of occurrence is often referred to as prevalence. For example, if one of the preferred criterion 110, 111, 112 is systolic blood pressure, information relating to the prevalence of systolic blood pressure levels, and to the levels used as “cut-points” or limits in classifying an individual risk as standard or preferred, has to be captured. For acquiring the prevalence relating to the risk exposed individuals 31, 32, 33, a large laboratory dataset of risk exposed individuals 31, 32, 33 are analyzed and filtered to select relevant prevalence information related, for example, to systolic blood pressure. The prevalence of preferred criteria 110, 111, 112 is then determined within an insured cohort. A cohort is a risk classification 101, 102, 103, which represents a range of incremental probabilities of occurrence of a life risk event. Therefore, the operation is a determination of the rate of occurrence of the subject criteria 110, 111, 112 among the members of a particular risk classification 101, 102, 103. To exclude correlations, which may exist among various ones of the preferred criteria 110,111, 112, i.e. a possible dependence between two or more of the preferred criteria 110, 111, 112, it may be reasonable to implement appropriate steps in the operation of the automated mortality classification system 1. For the determination of prevalence for all combinations of correlated criteria 110, 111, 112, a numerical representation of the prevalence within a population can e.g. be determined for each unique combination of criteria 110, 111, 112. If particular combinations of criteria 110, 111, 112 result in non-credible or aberrant results, adjustments must be made accordingly. From the representation, a probability of occurrence can be determined for each combination of criteria 110, 111, 112. The results of this determination can then be compared to the empirical data. If the prevalence of certain combinations varies with what can be analyzed form the empirical data, adjustments are made to match the empirical data. However, if this adjustment process results in anomalies, such anomalies can be detected and corrected by additional steps. When the prevalence results match the empirical evidence, the prevalence results are stored. The prevalence results for each combination of preferred criteria 110, 111, 112 are stored to the system 1 by issue age, gender, smoking, status, and duration etc.

Another process, important to the system 1, is the process for characterizing risks. This process can be performed before and/or contemporaneously to the operation of the automated mortality classification system 1, allowing a dynamic adaption of the system 1. This process relates to relative mortality (i.e., rate of death among preferred classes 101, 102, 103 divided by standard mortality). In a first step, data are captured from historical mortality data or other sources. This data includes data specific to each of the preferred criteria 110, 111, 112 being considered by the automated mortality classification system 1. In addition, other clinical/epidemiological data possibly available in connection with the preferred criteria 110, 111, 112 can be analyzed. Based on the analysis, a relative mortality rate for each of the criteria 110, 111, 112 can be calculated. As in the case with prevalence data, correlations in mortality data among the various criteria 110, 111, 112 should also be considered by the system 1. Finally, relative mortality rates are determined for all combinations of correlated criteria 110, 111, 112. Following these operations, any anomalies in the data have to be identified and resolved. The relative mortality rates determined for the combinations are compared with empirical data or data from studies, e.g. clinical studies, to determine whether the rates match the empirical data. Again, if the determined rates do not match the empirical evidence, adjustments to the relative mortality rates have to be made to match the empirical results by the system 1. Following possible adjustments, the data are checked or filtered for anomalies and any anomalies that occur are detected and corrected. If the relative mortality data is consistent with the empirical data or the data from studies, the data are stored by the automated mortality classification system 1. Accordingly to the prevalence, the relative, mortality results are stored to the system 1 for each correlated, preferred combination by issue age, gender, smoking status and duration etc. Finally, based on the prevalence and relative mortality results for each correlated combination of preferred criteria, a specific base-preferred criteria set 110, 111, 112 is selected by the automated mortality classification system 1. The selection can by conducted by the system 1 autonomously and/or e.g. negotiated in an automated way between the first and second insurance system 2/3. Based on the base criteria 110,111, 112, prevalence and relative mortality data can be extracted from the stored parameters for each of such criteria 110, 111, 112, and a relative risk ratio can be generated by the automated mortality classification system 1 for each risk class 101, 102,103 e.g. by age, gender and duration etc. The generation for each risk class 101, 102, 103 are based on both prevalence and relative mortality data, as well as on the preferred criteria 110, 111, 112 defining each risk class 101, 102, 103.

1. An automated, distinct-channel-based automated mortality classification and signaling system for real-time risk-assessment and adjustment, risks associated with a plurality of risk exposed individuals being at least partially transferable from a risk exposed individual to a first insurance system and/or from the first insurance system to an associated second insurance system, the system including a table with retrievable stored risk classes each comprising assigned risk class criteria, individual-specific parameters of the risk exposed individuals being captured relating to criteria of the stored risk classes by the system and stored in a memory, a specific risk class associated with the risk of the exposed individual being identified out and selected of the stored risk classes by the system based on the captured parameters, the system comprising:
a user interface via which a first channel, a second channel, and a third channel are selectable; and
processing circuitry configured to
in the first channel for each of the risk classes of the table with retrievable stored risk classes, determine and assign a first tolerance factor to the corresponding risk class, the criteria and/or related measuring parameters being dynamically adapted based on time-correlated incidence data for a preferred life risk condition indicating changes in the condition of the risk exposed individuals,
when the captured parameters of a risk exposed individual fail to be matched to the criteria for one of the retrievable stored risk classes by the system, generate and compare a relative mortality factor of the individual of the parameters to an average mortality of the closest matched class, and based on the assigned first tolerance factor of the closest matched class, indicate whether to accept or reject a possible risk-transfer for the individual for the closest matched class,
in the second channel, define class category parameters with assigned class category criteria comprising at least three class categories “standard,” “preferred,” and “better preferred,” measure a relative mortality factor based on the captured individual's specific parameter and the class category criteria and in relation to an average of expected mortality for the specific risk class associated with the individual, determine and assign a second tolerance factor for an excess mortality to the corresponding risk class, and indicate whether to assign the individual to the class category parameter “standard” or to a better class category “preferred” or “better preferred” based on the second tolerance factor, thereby providing the movement of the individual from class category “standard” to better class category factors “preferred” and/or “better preferred,”
in a third channel, capture individual-specific parameter, trigger for predefined decline parameters in the captured individual-specific parameter, and upon detecting one of the predefined decline parameters, decline a possible risk-transfer for the individual far any of the risk classes by transmitting appropriate decline data, and
based on the real-time risk-assessment and adjustment, transfer specific risks associated with the risk-exposed individuals from the risk-exposed individual to the first insurance system and/or from the first insurance system to the associated second insurance system, and generate and transmit an appropriate activation signaling to the first insurance system and/or to the associated second insurance system, the risk transfer being mutually synchronized between the first insurance system and second insurance system.

2. The system according to claim 1, wherein
by triggering predefined decline parameter in the captured individual-specific data, additional individual-specific parameters are requested and transmitted to an independent review entity, and
only upon capturing the transmission of a check back confirmation of the review entity, the processing circuitry declines a possible risk transfer for the individual for the classes by transmitting the appropriate decline data.

3. The system according to claim 1, wherein the processing circuitry at least partially transfers the risks associated with a plurality of risk exposed individuals on a facultative basis from a risk exposed individual to the first insurance system and/or from the first insurance system to the associated second insurance system.

4. The system according to claim 3, further comprising:
an input device provided in response to selection of a fourth channel for a facultative case summary submission for a new risk exposed individual, the fourth channel being selectable via the user interface when the captured parameters of the new risk exposed individual are not yet classified, the input device prompting for the new risk exposed individual of the first insurance system to enter facultative case summary information including risk factor information for evaluating a risk in providing risk cover by the second insurance system for the new risk exposed individual, wherein in response to received input, the processing circuitry is configured to render a facultative decision based on the received facultative case summary submission.

5. The system according to claim 1, wherein
when the captured parameters of a risk exposed individual are not yet classified, a freeform third channel extension is selectable via the user interface, the risk exposed individual being classified based upon matching to the parameters for one of the retrievable stored risk classes by the system, and
the first channel and/or the second channel and/or the third channel are only selectable via the user interface, when the captured parameters of a risk exposed individual fail to be matched to the criteria for one of the retrievable stored risk classes.

6. The system according to the claim 1, wherein
each of the risk classes of the table with retrievable stored risk classes is associated to at least one financial product accessible in a dedicated data store,
the processing circuitry is configured to determine, for each of the risk classes, an expected occurrence rate, divide the expected occurrence rates by an average rate and determine a relative risk ratio as relative mortality factor for each of the risk classes based on the data relating to the criteria associated with the risk classes, calculate correlated risk ratios between at least two of the risk classes that are identified, and determine a dependence between the at least two different risk classes, compare the relative risk ratios and the correlated risk ratios with empirical data and generate comparative risk data to characterize the relative risks associated with the plurality of products, correct the relative risk ratios when the comparative risk data is out of a defined range compared with the empirical data, and output the corrected risk ratios.

7. The system according to claim 1, wherein
the first channel comprises a table with retrievable stored impairment criteria, the first channel being only activatable in case of triggering at least one of the stored impairment criteria within the captured parameters of the risk exposed individuals resulting in a failure to be matched to one of the risk classes, and
the table with retrievable stored impairment criteria comprises as trigger criteria for medical impairment measuring parameters anemia, anxiety, asthma, atrial fibrillation and flutter, atrial septal defect, barrett's esophagus, bicuspid aortic valve, blood pressure, build, combination of blood pressure and lipids, combination of build and blood pressure, combination of build and lipids, crohn's disease, depression, diabetes mellitus type 2, epilepsy, mitral insufficiency, obstructive sleep apnea, rheumatoid arthritis, skin tumors other than melanoma, surgical treatment of obesity, thyroid, ulcerative colitis; comprises as trigger criteria for medical test criteria cholesterol/HDL ratio, EBCT, glomerular filtration rate (isolated elevation), EKG-T wave changes, impaired glucose tolerance, liver enzymes (isolated elevation), microalbuminuria (isolated elevation), proteinuria (isolated elevation), triglycerides; and comprises as trigger criteria for non-medical impairments aviation (private), driving, foreign travel, occupation, scuba diving.

8. The system according to claim 1, wherein
the second channel comprises a table with retrievable stored improvement criteria, the individual-specific parameters additionally comprise at least one of the retrievable stored improvement criteria, and
the improvement criteria comprise key preferred measuring parameters associated with the build, blood pressure and treatment, cholesterol and ratio and treatment, family history, glycohemoglobin, statins treatment, prevention, wellness, NT-proBNP, ECG, stress test, and/or EBCT of the risk exposed individual.

9. The system according to claim 1, wherein the processing circuitry at least partially transfers risks associated with the plurality of risk exposed individuals from the risk exposed individual to the first insurance system and/or from the first insurance system to the associated second insurance system, and generates and transmits the appropriate activation signaling to the first insurance system and/or to the associated second insurance system.

10. The system according to claim 1, wherein the processing circuitry is configured to automatically negotiate the risk class criteria between the first insurance system and the second insurance system.

11. The system according to claim 1, wherein the one or more risk classes are associated with one or more risk class criteria, and the processing circuitry is configured to further modify one or more of the criteria, re-determine the relative risk ratio, and determine an impact of the modification on the relative risks associated with the products.

12. The system according to claim 1, wherein the one or more of the risk classes are associated with different criteria, and the processing circuitry is further configured to compare the risk classes based on the relative risk ratios.

13. The system according to claim 12, wherein the processing circuitry is further configured to redefine one or more of the risk classes based on the relative risk ratios.

14. The system according to claim 12, wherein the processing circuitry is further configured to determine a separate relative risk ratio for sub-groups of risks.

15. The system according to claim 14, wherein the processing circuitry is further configured to compare prevalence data to industry empirical data for particular combinations of risk class criteria, and adjust the stored data to agree with the empirical data.

16. A distinct-channel-based automated mortality classification and signaling method for real-time risk-assessment and adjustment, wherein risks associated with a plurality of risk exposed individuals are at least partially transferred from a risk exposed individual to a first insurance system and/or from the first insurance system to an associated second insurance system, wherein an automated mortality classification system comprises a table with retrievable stored risk classes each comprising assigned risk class criteria, wherein individual-specific parameters of the risk exposed individuals are captured relating to criteria of the stored risk classes by the system and stored to a memory and wherein a specific risk class associated with the risk of the exposed individual is identified out and selected of said stored risk classes by the system based on the captured parameters, the method comprising:
in a first channel to each of the risk classes of the table with retrievable stored risk classes, determining and assigning a first tolerance factor to the corresponding risk class, the criteria and/or related measuring parameters being dynamically adapted based on time-correlated incidence data for a preferred life risk condition indicating changes in the condition of the risk exposed individuals, the first channel being selectable via a user interface;
when the captured parameters of a risk exposed individual fail to be matched to the criteria for one of the retrievable stored risk classes by the system, generating and comparing a relative mortality factor of the individual of the parameters to an average mortality of the closest matched class, and based on the assigned first tolerance factor of the closest matched class, indicating whether to accept or reject a possible risk-transfer for the individual for the closest matched class;
in a second channel, selectable via the user interface, defining class category parameters with assigned class category criteria comprising at least three class categories “standard,” “preferred” and “better preferred,” measuring a relative mortality factor based on the captured individual's specific parameter and the class category, criteria and in relation to the average of expected mortality for the specific risk class associated with the individual, determining and assigning a second tolerance factor for an excess mortality to the corresponding risk class, and indicating whether to assign the individual to the class category, parameter “standard” or to a better class category, “preferred” or “better preferred” based on the second tolerance factor, thereby providing the movement of the individual from class category “standard” to better class category factors “preferred” and/or “better preferred;”
in a third channel, selectable via the user interface, capturing individual-specific parameter, triggering for predefined decline parameters in the captured individual-specific parameter, and upon detecting one of the predefined decline parameters, declining a possible risk-transfer for the individual for any of the risk classes by transmitting appropriate decline data; and
based on the real-time risk-assessment and adjustment, transferring specific risks associated with the risk-exposed individuals from the risk-exposed individual to the first insurance system and/or from the first insurance system to the associated second insurance system, and generating and transmitting an appropriate activation signaling to the first insurance system and/or to the associated second insurance system, the risk transfer being mutually synchronized between the first insurance system and second insurance system.

17. The method according to claim 16, further comprising:
by triggering predefined decline parameter in the captured individual-specific data, requesting and transmitting additional individual-specific parameters to an independent review entity; and
only upon capturing the transmission of a check bock confirmation of the review entity, declining a possible risk transfer for the individual for the classes by transmitting appropriate decline data.

18. The method according to claim 16, further comprising;
at least partially transferring the risks associated with a plurality of risk exposed individuals on a facultative basis from a risk exposed individual to the first insurance system and/or from the first insurance system to the associated second insurance system.

19. The method according to claim 18, further comprising:
providing an input device in response to selection of a fourth channel for a facultative case summary submission for a new risk exposed individual, the fourth channel being selectable via the user interface when the captured parameters of the new risk exposed individual are not yet classified;
prompting, by the input device, for the new risk exposed individual of the first insurance system to enter facultative case summary information including risk factor information for evaluating a risk in providing risk cover by the second insurance system for the new risk exposed individual; and
in response to received input, rendering a facultative decision based on the received facultative case summary submission.

20. The method according to claim 16, wherein
when the captured parameters of a risk exposed individual ore not yet classified, a freeform third channel extension is selectable via the user interface, the risk exposed individual being classified based upon matching to the parameters for one of the retrievable stored risk classes by the system, and
the first channel and/or the second channel and/or the third channel are only selectable via the user interface, when the captured parameters of a risk exposed individual fail to be matched to the criteria for one of the retrievable stored risk classes.

21. The method according to claim 16, wherein each of the risk classes of the table with retrievable stored risk classes is associated to at least one financial product accessible in a dedicated data store, the method further comprising:
determining, for each of the risk classes, an expected occurrence rate, dividing the expected occurrence rates by an average rate and determining a relative risk ratio as relative mortality factor for each of the risk classes based on the data relating to the criteria associated with the risk classes, calculating correlated risk ratios between at least two of the risk classes that are identified, and determining a dependence between the at least two different risk classes, comparing the relative risk ratios and the correlated risk ratios with empirical data and generating comparative risk data to characterize the relative risks associated with the plurality of products; correcting the relative risk ratios when the comparative risk data is out of a defined range compared with the empirical data, and outputting the corrected risk ratios.

22. The method according to claim 16, wherein
the first channel comprises a table with retrievable stored impairment criteria, the first channel being only activatable in case of triggering at least one of the stored impairment criteria within the captured parameters of the risk exposed individuals resulting in a failure to be matched to one of the risk classes, and
the table with retrievable stored impairment criteria comprises as trigger criteria for medical impairment measuring parameters anemia, anxiety, asthma, atrial fibrillation and flutter, atrial septal defect, barrett's esophagus, bicuspid aortic valve, blood pressure, build, combination of blood pressure and lipids, combination of build and blood pressure, combination of build and lipids, crohn's disease, depression, diabetes mellitus type 2, epilepsy, mitral insufficiency, obstructive sleep apnea, rheumatoid arthritis, skin tumors other than melanoma, surgical treatment of obesity, thyroid, ulcerative colitis; comprises as trigger criteria for medical test criteria cholesterol/HDL ratio, EBCT, glomerular filtration rate (isolated elevation), EKG-T wave changes, impaired glucose tolerance, liver enzymes (isolated elevation), microalbuminuria (isolated elevation), proteinuria (isolated elevation), triglycerides; and comprises as trigger criteria for non-medical impairments aviation (private), driving, foreign travel, occupation, scuba diving.

23. The method according to claim 16, wherein
the second channel comprises a table with retrievable stored improvement criteria, the individual-specific parameters additionally comprise at least one of the retrievable stored improvement criteria; and
the improvement criteria comprise key preferred measuring parameters associated with the build, blood pressure and treatment, cholesterol and ratio and treatment, family history, glycohemoglobin, statins treatment, prevention, wellness, NT-proBNP, ECG, stress test, and/or EBCT of the risk exposed individual.

24. The method according to claim 16, further comprising:
at least partially transferring risks associated with the plurality of risk exposed individuals from the risk exposed individual to the first insurance system and/or from the first insurance system to the associated second insurance system; and
generating and transmitting the appropriate activation signaling to the first insurance system and/or to the associated second insurance system.

25. The method according to claim 16, further comprising:
automatically negotiating, by the processing circuitry, the risk class criteria between the first insurance system and the second insurance system.

26. The method according to claim 16, wherein the one or more risk classes are associated with one or more risk class criteria, the method further comprising:
further modifying one or more of the criteria, re-determining the relative risk ratio, and determining an impact of the modification on the relative risks associated with the products.

27. The method according to claim 16, wherein the one or more of the risk classes are associated with different criteria, the method further comprising:
comparing, by the processing circuitry, the risk classes based on the relative risk ratios.

28. The method according to claim 27, further comprising:
redefining one or more of the risk classes based on the relative risk ratios.

29. The method according to claim 27, further comprising:
determining a separate relative risk ratio for sub-groups of risks.

30. The method according to claim 29, further comprising:
comparing prevalence data to industry empirical data for particular combinations of risk class criteria, and adjusting the stored data to agree with the empirical data.
